(19) United States
(12) Patent Application Publication
(10) Pub. No.: US 2025/0195881 A1
(43) Pub. Date: Jun. 19, 2025

(54) ELECTRIC FIELD GENERATION DEVICE FOR INHIBITING TUMOR CELLS AND SYSTEM

(71) Applicant: SAFE CARE (SHAOXING) MEDICAL TECHNOLOGY CO., LTD., Shaoxing (CN)

(72) Inventors: Fusheng YOU, Shaoxing (CN); Hejun XUAN, Shaoxing (CN); Qibo XUAN, Shaoxing (CN)

(73) Assignee: SAFE CARE (SHAOXING) MEDICAL TECHNOLOGY CO., LTD., Shaoxing (CN)

(21) Appl. No.: 19/065,780

(22) Filed: Feb. 27, 2025

Related U.S. Application Data
(63) Continuation of application No. PCT/CN2023/124399, filed on Oct. 13, 2023.

(30) Foreign Application Priority Data
Aug. 29, 2022 (CN) 202211036743.5

(51) Int. Cl. A61N 1/36; A61N 1/04; A61N 1/372

(52) U.S. Cl. CPC A61N 1/36002; A61N 1/0476; A61N 1/3727

(57) ABSTRACT

An electric field generation device for inhibiting tumor cells and a system are provided. The electric field generation device includes a signal generation module, a waveform modulation module, a filter module, and an excitation module which are connected in sequence. The signal generation module is configured for generating a target waveform matched with a target electric field signal expected to be output. The waveform modulation module is configured for modulating the target waveform into a pulse sequence. The filter module is configured for carrying out low-pass filtering on the pulse sequence and converting the pulse sequence into the target electric field signal with the same type and frequency as the target waveform. The excitation module is configured for applying the target electric field signal on tumor cells.

### CROSS-REFERENCE TO RELATED APPLICATIONS

This application is a continuation of international patent application No. PCT/CN2023/124399,filed on Oct. 13, 2023, which itself claims priority to Chinese patent application No. 202211036743.5, filed on Aug. 29, 2022, titled “ELECTRIC FIELD GENERATION DEVICE FOR INHIBITING TUMOR CELLS AND SYSTEM”. The contents of the above identified applications are hereby incorporated herein in their entireties by reference.

### TECHNICAL FIELD

The present disclosure generally relates to the field of medical devices, and in particular, to an electric field generation device for inhibiting tumor cells and a system.

### BACKGROUND

Cell therapy with electric fields, also known as Tumor-Therapeutic Fields (TTF), is a new type of cell therapy. Cell therapy with electric fields can output low-intensity, medium-frequency, alternating electric fields in a target tissue area, interfere with a cell division process, cause cell death, and achieve a therapeutic goal. In other words, a basic principle of electric field therapy is based on the inhibitory and destructive effect of electric field on tumor cell mitosis. Generally, an electric field generated by an electrical signal with a frequency of 200 KHz can be used to inhibit rapid growth of tumor cells in patients, in order to achieve therapeutic effects.

In the related art, a single waveform type of an electric field signal is often used to act on tumor cells, but the ability of a single waveform has certain limitations and cannot produce good inhibition effects on all types of tumor cells.

### SUMMARY

According to various embodiments of the present disclosure, an electric field generation device for inhibiting tumor cells and a system thereof are provided.

In a first aspect, the electric field generation device for inhibiting the tumor cells is provided in an embodiment of the present disclosure. The electric field generation device includes a signal generation module, a waveform modulation module, a filter module, and an excitation module which are connected in sequence.

The signal generation module is configured for generating a target waveform matched with a target electric field signal expected to be output. A type and frequency of the target waveform are at least adjustable.

The waveform modulation module is configured for modulating the target waveform into a pulse sequence. A width of a pulse in the pulse sequence is matched with an amplitude of the target waveform.

The filter module is configured for carrying out low-pass filtering on the pulse sequence and converting the pulse sequence into the target electric field signal with the same type and frequency as the target waveform.

The excitation module is configured for applying the target electric field signal on tumor cells.

Alternatively, in an embodiment of the present disclosure, the waveform modulation module includes a modulation circuit and an inverter. The inverter may include a plurality of switching devices. An output terminal of the modulation circuit has a signal connection to the plurality of switching devices in the inverter.

The modulation circuit is configured to take the target waveform as a modulated wave, and control on/off of the plurality of switching devices in the inverter according to intersection points between the target waveform and a carrier wave, so that the inverter is capable of outputting the pulse sequence. The width of the pulse in the pulse sequence is matched with the amplitude of the target waveform.

Alternatively, in an embodiment of the present disclosure, a power supply of the inverter is adjustable. A voltage value of the power supply is matched with an excitation voltage value required for inhibiting the tumor cells.

Alternatively, in an embodiment of the present disclosure, the signal generation module is further configured to generate a differential waveform with the target waveform while generating the target waveform. Correspondingly, the modulation circuit is further configured to control the on/off of different switching devices in the inverter according to the intersection points between the target waveform and the carrier wave and intersection points between the differential waveform and the carrier wave, so that the inverter is capable of outputting two pulse sequences, a width of the pulse in one pulse sequence is matched with the amplitude of the target waveform, and a width of the pulse in the other pulse sequence is matched with the amplitude of the differential waveform.

Alternatively, in an embodiment of the present disclosure, the filter module includes a selection circuit and at least one filter circuit. Each filter circuit is corresponding to one type of waveform.

The selection circuit is configured to input the pulse sequence to a filter circuit matched with the type of the target waveform in response to a received selection signal.

Alternatively, in an embodiment of the present disclosure, the filter module further includes a transformer that is configured to perform voltage boosting on the electric field signal after filtering to generate the target electric field signal.

Alternatively, in an embodiment of the present disclosure, the excitation module includes an electrode selection channel and at least one group of electrodes. The electrode selection channel is connected to each group of electrodes one by one and is configured to control whether to transmit the target electric field signal to each group of electrodes. The at least one group of electrodes is configured to receive the target electric field signal to form a spatial electric field that acts on the tumor cells.

Alternatively, in an embodiment of the present disclosure, the at least one group of electrodes is arranged oppositely along different directions to receive the target electric field signal to form a spatial electric field which is capable of penetrating a tumor region.

In a second aspect, a system for inhibiting tumor cells is provided in an embodiment of the present disclosure. The system includes a controller and the electric field generation device. The controller is signal-coupled with the electric field generation device and configured to adjust the type and frequency of the target waveform.

Alternatively, in an embodiment of the present disclosure, the system further includes a user terminal. The user terminal is signal-coupled with the controller and configured to receive a control instruction input by a user and transmit the control instruction to the controller.

Details of one or more embodiments of the present disclosure are proposed in the following accompanying drawings and descriptions, so that other features, objects, and advantages of the present disclosure are more easily understood.

### BRIEF DESCRIPTION OF THE DRAWINGS

In order to more clearly describe technical solutions in the embodiments or the related art, the following briefly describes the accompanying drawings required for describing the embodiments or the conventional technologies. Apparently, the accompanying drawings in the following description show merely the embodiments of the present disclosure, and one skilled in the art may still derive other drawings from the disclosed accompanying drawings without creative efforts.

FIG. 1 is a schematic diagram of a module of an electric field generation device 100 in an embodiment.

FIG. 2 is a comparative diagram of sine half wave and PWM waveform in an embodiment.

FIG. 3 is a schematic diagram of a module structure based on bipolar modulation in an embodiment.

FIG. 4 is a schematic diagram of a waveform based on bipolar modulation in an embodiment.

FIG. 5 is a schematic diagram of a module structure based on frequency multiply modulation in an embodiment.

FIG. 6 is a schematic diagram of a waveform based on frequency multiply modulation in an embodiment.

FIG. 7 is a schematic diagram of a filter module and an excitation module in an embodiment.

FIG. 8 is a schematic diagram of a module of a system 800 for inhibiting the tumor cells in an embodiment.

### DETAILED DESCRIPTION OF THE EMBODIMENT

The following will provide a clear and complete description of the technical solution in the embodiments of the present disclosure, in communication with the accompanying drawings. Obviously, the described embodiments are only a part of the embodiments of the present disclosure, not all of them. Based on the embodiments in the present disclosure, all other embodiments obtained by ordinary skill in this art without creative labor fall within the scope of protection of the present disclosure.

Exemplary embodiments will be described in detail herein, with the examples illustrated in the accompanying drawings. When the description involves the drawings, unless otherwise indicated, the same numbers in different drawings represent the same or similar elements. Implementation methods described in the following exemplary embodiments do not represent all the implementation methods consistent with the present disclosure. On the contrary, they are merely examples of devices consistent with some aspects of the present disclosure as detailed in the appended claims.

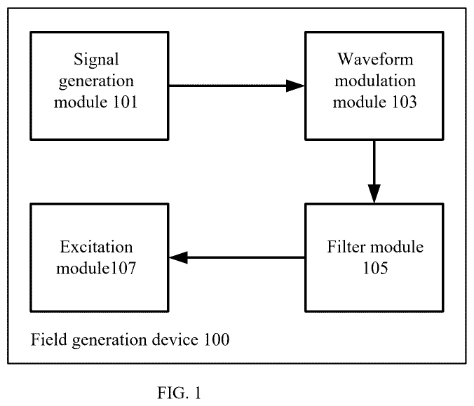

To facilitate understanding of technical solutions provided in the embodiments of the present disclosure for one skilled in the art, a technical environment in which the technical solutions are implemented will first be described with reference to FIG. 1.

In the related art, it has been proposed to use electric field signals of a single waveform type to stimulate the tumor cells. However, such electric field signals of the single waveform type have certain limitations and are not applicable to all types of the tumor cells. In the embodiments of the present disclosure, an electric field generation device capable of producing electric field signals of multiple different waveform types is proposed. This electric field generation device not only can use electric field signals with composite waves to inhibit the tumor cells, not limited to conventional waveform types such as a sine wave, a triangular wave, a square wave, and a sawtooth wave, but can also produce electric field signals of any desired waveform type, which is not limited herein.

FIG. 1 is a schematic diagram of an electric field generation device 100 for inhibiting tumor cells. Referring to FIG. 1, an electric field generation device 100 includes a signal generation module 101, a waveform modulation module 103, a filter module 105, and an excitation module 107 which are connected in sequence.

The signal generation module 101 is configured for generating a target waveform matched with a target electric field signal expected to be output. A type and frequency of the target waveform are at least adjustable.

The waveform modulation module 103 is configured for modulating the target waveform into a pulse sequence, and a width of a pulse in the pulse sequence is matched with an amplitude of the target waveform.

The filter module 105 is configured for carrying out low-pass filtering on the pulse sequence and converting the pulse sequence into the target electric field signal with the same type and the same frequency as the target waveform.

The excitation module 107 is configured for applying the target electric field signal on the tumor cells.

In the present embodiment, the signal generation module is configured for generating the target waveform matched with the target electric field signal expected to be output, and the type and the frequency of the target waveform are at least adjustable. In a real application, the signal generation module 101 may generate a target waveform according to an instruction of waveform switch, for example, an input terminal of the signal generation module 101 may be electrically connected to a processor (such as MCU (Microcontroller Unit)). The processor may send a waveform switching instruction to the signal generation module 101, such that the signal generation module 101 may be indicated to generate any type of the target waveform, and parameters of the target waveform such as a frequency, an amplitude and so on may also be set. Alternatively, in an embodiment of the present disclosure, the signal generation module 101 may include a Direct Digital Synthesizer (DDS), the DDS may generate various types of waveforms such as sine waves, triangular waves, square waves, sawtooth waves and so on. It not only has the advantages of low cost, low power consumption, and high resolution, but also has the advantage of fast switching. Therefore, it can achieve fast switching of multiple waveform types or different waveform frequencies in composite waves, which meets efficient requirements of a process of inhibiting the tumor cells. Alternatively, in other embodiments, the signal generation module 101 may further include any signal generator capable of generating multi-waveform signals, such as a function signal generator, which is not limited herein.

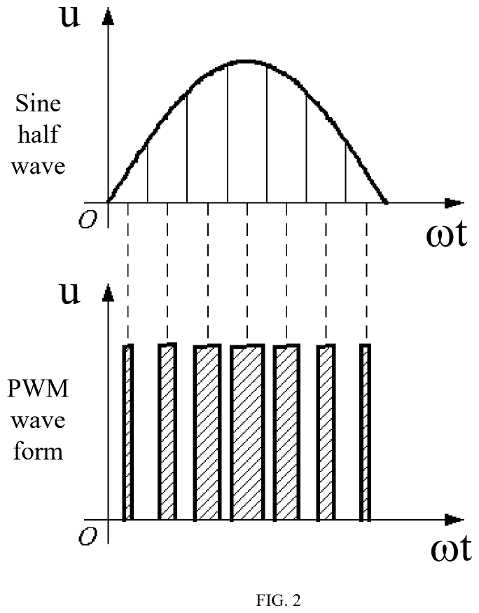

In real applications, for example, for stimulating tumor cells in the human brain, an electric field signal with a peak-to-peak value of 20 V to 100 V may often be required according to a size of the brain, with a minimum of 1.0 Vpp/cm applied to the tumor cells. However, the signal generated by the signal generator may often be weak and insufficient to drive components such as electrodes that act on the tumor cells. For example, the DDS may use a signal source with an output level in mA range, so an amplitude of an output target waveform may often be relatively small, which is not on the same order of magnitude as the strength of the electric field signal required to stimulate the tumor cells. The electric field generating device 100 provided in various embodiments of the present disclosure generates the target electric field signal with the same type and frequency as the target waveform based on the target waveform, but the energy of the target electric field signal may be much higher than that of the target signal generated by the signal generator. In the embodiments of the present disclosure, the waveform modulation module 103 is configured for modulating the target waveform into the pulse sequence, and the width of the pulse in the pulse sequence is matched with the amplitude of the target waveform. The pulse sequence is converted into the target electric field signal acted on the tumor cells. The pulse sequence may include a pulse width modulation (PWM) waveform. The width of the pulse in the PWM may be matched with the amplitude of the target waveform, which is illustrated by an example. Referring to FIG. 2, the sine half wave in FIG. 2 may be divided into N equal parts, so the sine half wave (duration T) may be equivalent to a waveform including N pulse connected to each other. Widths of the pulses are equal (all equal to T/N), but amplitudes of the pulses may not be equal, and a top of the pulses may not be a horizontal straight line, but a curve. The amplitudes of pulses may vary sinusoidally. When the above pulses are replaced with an equal number of rectangular pulses with equal amplitude but not equal width, a midpoint of a rectangular pulse may coincide with a midpoint of a corresponding part of the sine wave, and an area of the rectangular pulse may be equal to that of the corresponding part of the sine wave, the pulse sequence shown in FIG. 2 may be obtained, which is the PWM waveform. Amplitudes of pulses in the pulse sequence may be equal, and widths of pulses in the pulse sequence may vary sinusoidally. According to a principle of area equivalence, the PWM waveform and the sine half wave may be equivalent.

In the embodiments of the present disclosure, during a process of modulating the target waveform into the pulse sequence, the target waveform may be modulated by a relatively high voltage. Therefore, the amplitude of the generated pulse sequence may be relatively high. After generating the pulse sequence, the pulse sequence may be carried out low-pass filtering by the filter module 105, for example, an appropriate cutoff frequency may be set to filter out harmonic components of a high-frequency signal in the pulse sequence, so as to generate the target electric field signal. The waveform type and frequency of the target electric field signal are the same as that of the target waveform. For example, the signal generation module 101 may generate a sine wave with a frequency of 200 KHz. After processing by the filter module 105, a waveform of the generated target electric field signal may also be a sine wave, and a frequency of the generated target electric field signal may also be 200 KHz, just an amplitude of the generated target electric field signal may be much greater than that of the target waveform. Furthermore, after generating the target electric field signal, the target electric field signal may be acted on the tumor cells by the excitation module 107, thereby inhibiting the tumor cells.

In the embodiments of the present disclosure, the electric field generation device 100 for inhibiting the tumor cells generate the target waveform matched with the target electric field signal expected to be output by the signal generation module 101. The type and the frequency of the target waveform may be adjusted. In this way, the type and the frequency of the target waveform may be freely adjusted when the electric field generation device 100 is acted on the tumor cells, so as to meet a requirement for multiple types of waveforms and multiple operating frequencies when stimulating the tumor cells, i.e., meeting a requirement for stimulating the tumor cells by composite waves. Since the amplitude of the target waveform provided by the signal generation module 101 is relatively low, which is not capable of driving electrodes configured to stimulate the tumor cells. Therefore, in the embodiments of the present disclosure, the target waveform is modulated into the pulse sequence by the waveform modulation module 103, the widths of the pulses in the pulse sequence may be set to be matched with the amplitude of the target waveform. In this way, a target waveform with low signal strength may be converted into the pulse sequence with relatively high signal strength. The pulse sequence may be carried out low-pass filtering by the filter module 105 to filter higher-order harmonics in the pulse sequence, such that the pulse sequence may be converted into the target electric field signal with the same type and frequency as the target waveform. The target electric field signal may be acted on the tumor cells finally.

In an embodiment of the present disclosure, the waveform modulation module 103 may include a modulation circuit and an inverter. The inverter may include a plurality of switching devices. An output terminal of the modulation circuit may have a signal connection to the plurality of switching devices in the inverter.

The modulation circuit is configured to take the target waveform as a modulated wave, and control on/off of the plurality of switching devices in the inverter according to intersection points between the target waveform and a carrier wave, so that the inverter is capable of outputting the pulse sequence, and the width of the pulse in the pulse sequence may be matched with the amplitude of the target waveform.

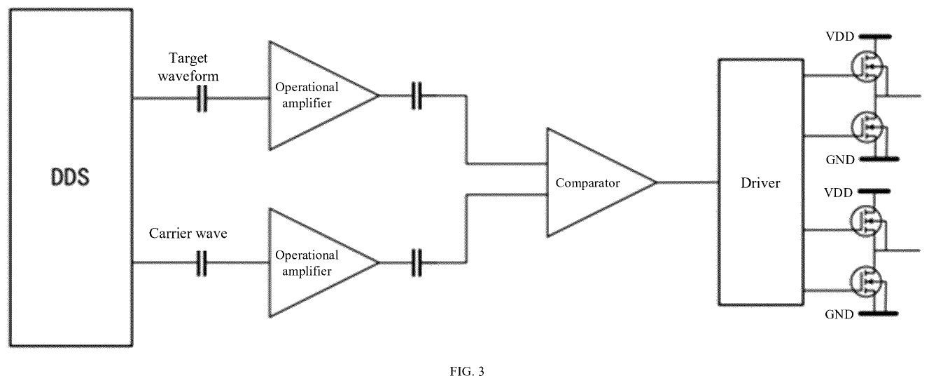

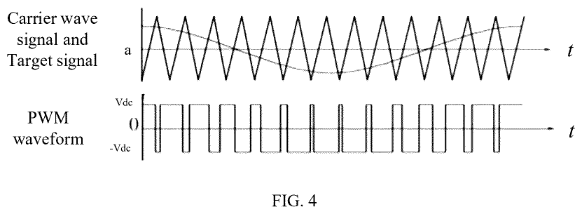

In the embodiments of the present disclosure, the waveform modulation module 103 may mainly include the modulation circuit and the inverter. A signal generated by the modulation circuit may be a controlling signal to control the on/off of the plurality of switching devices in the inverter. Specifically, FIG. 3 provides a schematic structure of the waveform modulation module 103. Referring to FIG. 3, the DDS may generate the target waveform and a carrier wave signal at the same time. The carrier wave signal may include an isosceles triangular wave, a frequency of which is much higher than that of the target waveform, for example, the frequency of the carrier wave signal may be six or more times than that of the target waveform. Referring to FIG. 3, since a signal generated by the DDS is relatively weak, the target waveform and carrier signal may be amplified by an operational amplifier. In addition, capacitors may be set before and after the operational amplifier to isolate a DC signal, so that a reference point of the target waveform may be the same as that of the carrier wave signal. In the embodiments of the present disclosure, the intersection points between the target waveform and the carrier wave may be determined according to a comparator. Referring to FIG. 3, an amplified target signal and an amplified carrier wave signal may be input into two terminals of the comparator, so as to determine comparison results of the amplitude of the target signal and the amplitude of the carrier wave signal at each moment. The inverter may include a plurality of switching devices, such as H-bridge structures. The plurality of switching devices may include a high-power transistor, such as a MOS power transistor. An input result of the comparator may determine the off/on of each of the plurality of switching devices, such that the inverter may output the PWM signal. In addition, referring to FIG. 3, an input result of the comparator may control the off/on of each of the plurality of switching devices by a driver. The driver may include a power driver, such that actions of off/on of the plurality of switching devices may be quick, i.e., increase and decrease of a driving current may be both quick, and delay of the target electric field signal may be reduced. The driver may include various types of drivers such as a voltage regulating driver, a voltage boosting driver, a capacitive driver, and so on, which are not limited herein. FIG. 4 shows a relationship among the carrier wave signal, the target signal, and a generated PWM waveform. Referring to FIG. 4, the intersection points between the target signal and the carrier wave may represent time of switching the off/on of each of the plurality of switching devices. Switching the off/on of the plurality of devices may cause that a voltage value output by the inverter switches between the highest value and the lowest value, and the PWM wave may be formed finally.

In an embodiment of the present disclosure, the signal generation module 101 is further configured to generate a differential waveform with the target waveform while generating the target waveform. Correspondingly, the modulation circuit is further configured to control the on/off of different switching devices in the inverter according to the intersection points between the target waveform and the carrier wave and intersection points between the differential waveform and the carrier wave, so that the inverter is capable of outputting two pulse sequences, a width of the pulse in one pulse sequence may be matched with the amplitude of the target waveform, and a width of the pulse in the other pulse sequence may be matched with the amplitude of the differential waveform.

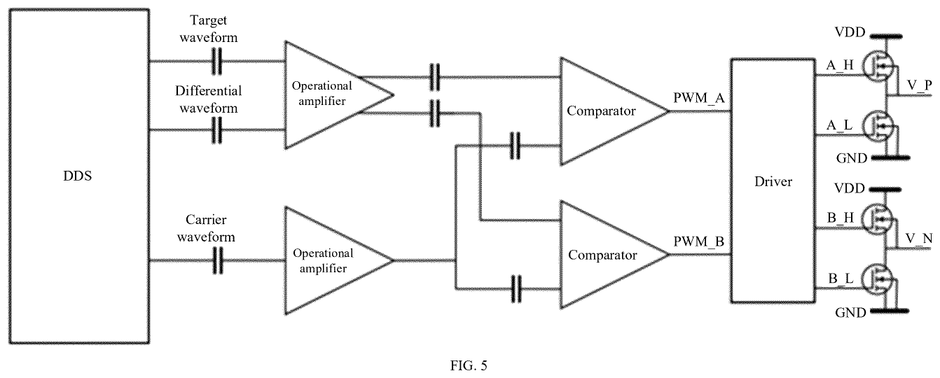

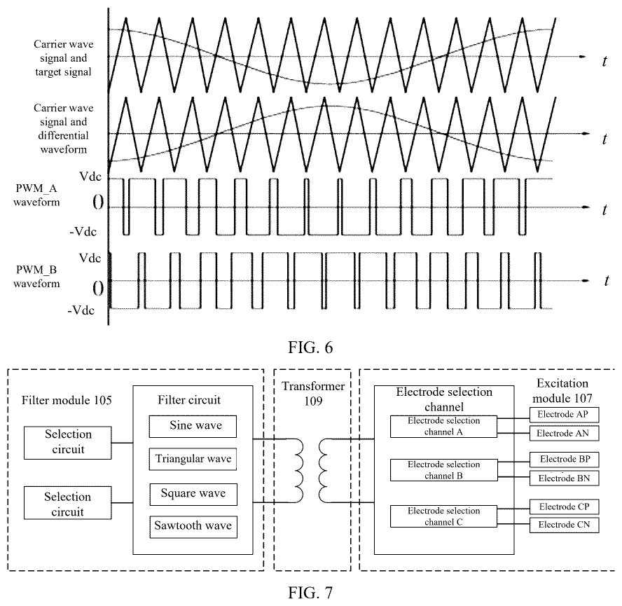

In the embodiments of the present disclosure, a phase difference between the differential waveform and the target waveform may be 180 degrees. FIG. 5 shows a specific implementation structure. Referring to FIG. 5, after the DDS generating the target waveform, the differential waveform and a carrier waveform, and the operational amplifier amplifying the corresponding signal, the target waveform may be compared with the carrier waveform, and the differential waveform may be compared with the carrier waveform. Similarly, the off/on of the plurality of switching devices in the inverter may be controlled by comparison results. FIG. 6 shows a schematic diagram of the target waveform and the carrier waveform, the differential waveform and the carrier waveform, and a PWM_A waveform and a PWM_B waveform. Specifically, during an operation process, after being driven by the driver, the PWM_A output by the comparator may generate a A_H wave and a A_L wave complementary to each other. After being driven by the driver, the PWM_B output by the comparator may generate a B_H wave and a B_L wave complementary to each other. The A_H wave, the A_L wave, the B_H wave and the B_L wave may be acted on a high end and a low end of the H-bridge, respectively, so as to generate a V_P wave and a V_N wave, in which voltage amplitudes of the V_P wave and the V_N wave may be VDD. The V_P wave may be a PWM wave that a pulse width thereof is the same as that of the PWM_A and an amplitude thereof is different from that of the PWM_A. The V_N wave may be a PWM wave that a pulse width thereof is the same as that of the PWM B and an amplitude thereof is different from that of the PWM_B.

In the embodiment of the present disclosure, the PWM waveform generated by modulating the target waveform and the complementary waveform is configured to control the on/off of the plurality of switching elements in the inverter. Compared with the PWM waveform generated by modulating the target waveform, the frequency of the former PWM controlling may be twice the frequency of the latter PWM controlling when the operating frequencies of the switching elements are the same, which may significantly reduce harmonics in the output waveform of the inverter. The subsequent filtering module 105 may require relatively small inductance or capacitance filtering devices to achieve good filtering effect.

In real applications, energy required to inhibit the tumor cells may vary depending on different parts of a human body, which means that voltage values of the applied electric field are different. In an embodiment of the present disclosure, a power supply of the inverter may be adjustable, and a voltage value of the power supply may be matched with an excitation voltage value required for inhibiting the tumor cells. By adjusting the voltage value of the power supply of the inverter, the target electric field signal with different peak-to-peak values may be generated. For example, for inhibiting brain tumor cells, the voltage value of the power supply may be adjusted to 50V, and for inhibiting tumor cells in other parts, the voltage value of the power supply may be adjusted to 60V. Alternatively, in another embodiment of the present disclosure, the target signal may be processed by an auxiliary adjustment of the signal generator. The adjustment of the signal generator for an amplitude of the target signal may influence an amplitude of a finally generated target electric field signal. Therefore, the adjustment of the power supply of the inverter may be a main adjustment, the adjustment of the signal generator for the target signal may be the auxiliary adjustment, so as to obtain an accuracy target amplitude and realize flexibly of the adjustment of the amplitude of the target electric field signal.

In real applications, for different types of waveforms, cutoff frequencies may be different during a filtering process. In an embodiment of the present disclosure, the filter module 105 may include a selection circuit and at least one filter circuit. Each filter circuit may correspond to one type of waveform.

The selection circuit is configured to input the pulse sequence to a filter circuit matched with the type of the target waveform in response to a received selection signal.

In the embodiment of the present disclosure, in the filter module 105, for example, a sine wave may correspond to a filter circuit, a square ware may correspond to a filter circuit, and a sawtooth wave may correspond to a filter circuit and so on, for different filter circuits, the cutoff frequency matched with the type of the target waveform may be set. In this way, filter processes corresponding to different waveforms may be flexibly achieved.

In an embodiment of the present disclosure, the filter module 105 may further include a transformer 109 that is configured to perform voltage boosting on the electric field signal after filtering to generate the target electric field signal. In the embodiment, the electric field signal may be boosted by the transformer 109. In this way, the power supply of the inverter in the waveform modulation module 103 may be appropriately reduced. The power supply of the inverter may be reduced, and a size of the switching device in the inverter may be reduced correspondingly, such that a size of the whole electric field generation device 100 may be reduced. Since the transformer 109 has a magnetic path and no electrical path, the transformer 109 may have an electrical isolation function, which may isolate high current at the input terminal from the output terminal, so that the target electric field signal that is output is a low current signal, preventing the high current signal from causing harm to the human body and improving personal safety protection for patients.

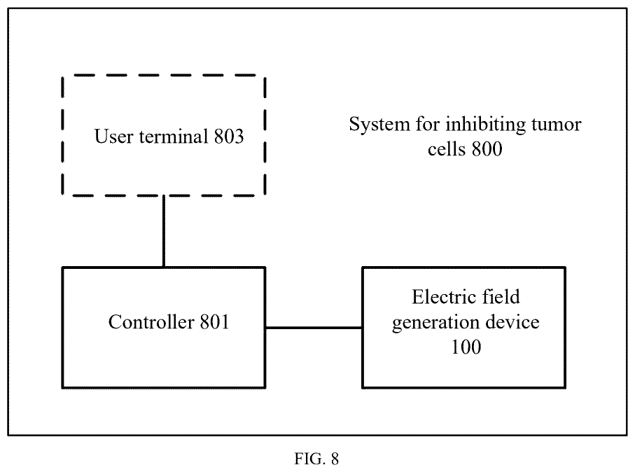

In an embodiment, the excitation module 107 may include an electrode selection channel and at least one group of electrodes. The electrode selection channel may be connected to each group of electrodes one by one and be configured to control whether to transmit the target electric field signal to each group of electrodes. The at least one group of electrodes is configured to receive the target electric field signal to form a spatial electric field that acts on the tumor cells. Referring to FIG. 7, three groups of the electrodes may be acted on the tumor cells. Each of three groups of the electrodes may be disposed on different positions of the human body.

In the embodiment of the present disclosure, a plurality of groups of the electrodes may be disposed in the excitation module 107, such that the plurality of groups of the electrodes may stimulate the tumor cells from different directions, respectively, thereby getting a better healing effect. In an embodiment, two groups of the electrodes with perpendicular directions may be alternately applied to the tumor cells. The electrode selection channel is configured to select an electrode corresponding to the electrode selection channel. In an embodiment, the electrode selection channel may include a controlling switch. The target electric field signal may be selected to transmit to a corresponding electrode by controlling off/on of the controlling switch. The controlling switch may include transistors, power field-effect transistors, thyristors, and so on, which are not limited herein.

In an embodiment of the present disclosure, the at least one group of electrodes may be arranged on a surface of the human body oppositely along different directions to receive the target electric field signal to form the spatial electric field which is capable of penetrating a tumor region. The electrodes may include patch-type electrodes. Placing the patch-type electrodes on the surface of the human body may have many technical advantages. In an aspect, the method may be convenient and fast, and may flexibly adjust a placement of the electrode on the surface of the human body. In another aspect, the method may not require to intervene in the human body and not cause pain to patients.

Alternatively, in other embodiments, since a method of intervening in the human body has a relatively high healing effect, based on this, the electrode may further include a probe. The probe may be intervened in the human body to come into contact with lesions or tumors and stimulate the lesions or the tumors, and a structure of the electrode is not limited herein.

In another aspect of the present disclosure, a system for inhibiting tumor cells 800 is provided. Referring to FIG. 8, the system 800 may include the electric field generation device 100 of any one of the above embodiments and a controller 801. The controller 801 is signal-coupled with the electric field generation device and configured to adjust the type and frequency of the target waveform. The controller 801 may control parameters such as duration of action for each type of waveforms, a specific operation method may be stored in a form of program code in a computer-readable storage medium within the controller 801. In this way, the controller 801 may implement the operation method by executing the program code.

The controller 801 may control the signal generation module 101 to output target waveforms with target types and/or target frequencies, and control numerous other modules within the electric field generation device 100. In an embodiment of the present disclosure, the controller 801 is configured to adjust the voltage value of the power supply of the inverter, such that the voltage value is matched with the excitation voltage required to inhibit the tumor cells. In another embodiment of the present disclosure, the controller 801 is configured to generate a selection signal and send the selection signal to the selection circuit of the filter module 105. In this way, the selection circuit may be in response to the selection signal, and input the pulse sequence into the filter circuit matched with the type of the target waveform. In a specific embodiment, when the target waveform is the sawtooth wave, the controller 801 may generate the selection signal corresponding to the sawtooth wave, such that the selection circuit may input the pulse sequence into the filter circuit matched with the sawtooth wave. In another embodiment of the present disclosure, the controller 801 is further configured to control the electrode selection channel of the excitation module 107. By controlling the electrode selection channel, a group of electrodes may be selected to be conducted or an incentive duration of each group of the electrodes may be adjusted.

In an embodiment of the present disclosure, the system 800 may further include a user terminal 803, the user terminal 803 may be signal-coupled with the controller 801 and configured to receive a control instruction input by a user and transmit the control instruction to the controller 801. The user terminal 803 may be wired or wireless connected to the controller 801, and the wireless connection may include Bluetooth, WIFI, and other connection methods. The user may input the control instruction on the user terminal 803, for example, the user may selects a waveform combination for exciting the tumor cells on the user terminal 803. After transmitting the control instruction to the controller 801, the controller 801 may perform the control instruction to exciting the tumor cells by the waveform combination. The user may send the control instruction to the electric field generation device 100 by the user terminal 803, so as to achieve interaction between the user and the electric field generating device 100, resulting in meeting a requirement of the user for custom excitation waveforms.

For the controller 801, the controller 801 may allow the user to program and integrate a digital system onto a single PLD, there is no requirement for chip manufacturers to design and manufacture specialized integrated circuit chips. At present, instead of manually making integrated circuit chips, programming may be mostly implemented by “logic compiler” software, which is similar to a software compiler used in program development and writing. An original code before compilation may also require to be written in a specific programming language, called Hardware Description Language (HDL). HDL may not be just one type, but there are many types, such as ABEL (Advanced Boolean Expression Language), AHDL (Altera Hardware Description Language), Confluence, CUPL (Cornell University Programming Language), HDCal, JHDL (Java Hardware Description Language), Lava, Lola, MyHDL, PALASM, RHDL (Ruby Hardware Description Language) and so on. Currently, the most commonly used programming languages are VHDL (Very-High-Speed Integrated Circuit Hardware Description Language) and Verilog. The skilled in the art should be clear that it is easy to obtain a hardware circuit that implements the logical method flow by simply programming the method flow with the above hardware description languages and programming the method flow into the integrated circuit.

For the controller 801, it may be achieved by any appropriate way. For example, the controller 801 may take the form of, for example, a microcontroller 801 or a controller 801, as well as computer-readable media, logic gates, switches, Application Specific Integrated Circuits (ASICs), programmable logic controllers, and embedded microcontrollers that store computer-readable program code (such as software or firmware) that can be executed by the (micro) controller 801. Examples of the controller 801 include but are not limited to the following microcontrollers: ARC 625D, Atmel AT91SAM, Microchip PIC18F26K20, and Silicone Labs C8051F320. A memory controller may also be implemented as part of the control logic of the memory. The skilled in the art should be clear that, expect for implementing the controller 801 in pure computer-readable program code, the same functionalities in the form of logic gates, switches, application specific integrated circuits, programmable logic controllers, and embedded microcontrollers may be entirely achieved by logically programming the method steps. Therefore, this controller 801 can be considered as a hardware component, and the devices included therein for implementing various functions may also be regarded as structures within the hardware component. Or even, devices configured to implement various functions may be regarded as both software modules for implementing methods and structures within hardware components.

The various embodiments described in this specification are presented in a progressive manner. The identical or similar parts among different embodiments can be referred to each other. Each embodiment focuses on the aspects that are different from the others. In particular, for the system embodiments, since the system embodiments are essentially similar to the method embodiments, the system embodiments are described in a relatively simplified manner. For related aspects, reference can be made to the description of the method embodiments.

The above description is merely illustrative of the embodiments of the present disclosure and is not intended to limit the present disclosure. For those skilled in the art, the present disclosure can be subject to various modifications and variations. Any modifications, equivalent substitutions, improvements, and the like made within the spirit and principles of the present disclosure should all be included within the scope of the claims of the present disclosure.

What is claimed is:

1. An electric field generation device for inhibiting tumor cells, comprising a signal generation module, a waveform modulation module, a filter module, and an excitation module which are connected in sequence, wherein
the signal generation module is configured for generating a target waveform matched with a target electric field signal expected to be output, and a type and frequency of the target waveform are at least adjustable;
the waveform modulation module is configured for modulating the target waveform into a pulse sequence, and a width of a pulse in the pulse sequence is matched with an amplitude of the target waveform;
the filter module is configured for carrying out low-pass filtering on the pulse sequence and converting the pulse sequence into the target electric field signal with the same type and frequency as the target waveform; and
the excitation module is configured for applying the target electric field signal on tumor cells.

2. The electric field generation device of claim 1, wherein the waveform modulation module comprises a modulation circuit and an inverter, the inverter comprises a plurality of switching devices, and an output terminal of the modulation circuit has a signal connection to the plurality of switching devices in the inverter; and
the modulation circuit is configured to take the target waveform as a modulated wave, and control on/off of the plurality of switching devices in the inverter according to intersection points between the target waveform and a carrier wave, so that the inverter is capable of outputting the pulse sequence, and the width of the pulse in the pulse sequence is matched with the amplitude of the target waveform.

3. The electric field generation device of claim 2, wherein a power supply of the inverter is adjustable, and a voltage value of the power supply is matched with an excitation voltage value required for inhibiting the tumor cells.

4. The electric field generation device of claim 2, wherein the signal generation module is further configured to generate a differential waveform with the target waveform while generating the target waveform, and correspondingly, the modulation circuit is further configured to control the on/off of different switching devices in the inverter according to the intersection points between the target waveform and the carrier wave and intersection points between the differential waveform and the carrier wave, so that the inverter is capable of outputting two pulse sequences, a width of the pulse in one pulse sequence is matched with the amplitude of the target waveform, and a width of the pulse in the other pulse sequence is matched with the amplitude of the differential waveform.

5. The electric field generation device of claim 1, wherein the filter module comprises a selection circuit and at least one filter circuit, each filter circuit is corresponding to one type of waveform, and the selection circuit is configured to input the pulse sequence to a filter circuit matched with the type of the target waveform in response to a received selection signal.

6. The electric field generation device of claim 1, wherein the filter module further comprises a transformer that is configured to perform voltage boosting on the electric field signal after filtering to generate the target electric field signal.

7. The electric field generation device of claim 1, wherein the excitation module comprises an electrode selection channel and at least one group of electrodes, the electrode selection channel is connected to each group of electrodes one by one and is configured to control whether to transmit the target electric field signal to each group of electrodes, the at least one group of electrodes is configured to receive the target electric field signal to form a spatial electric field that acts on the tumor cells.

8. The electric field generation device of claim 7, wherein the at least one group of electrodes is arranged oppositely along different directions to receive the target electric field signal to form a spatial electric field which is capable of penetrating a tumor region.

9. A system for inhibiting tumor cells, comprising a controller and the electric field generation device of claim 1, wherein the controller is signal-coupled with the electric field generation device and configured to adjust the type and frequency of the target waveform.

10. The system of claim 9, further comprising a user terminal, wherein the user terminal is signal-coupled with the controller and configured to receive a control instruction input by a user and transmit the control instruction to the controller.

11. The system of claim 9, wherein the waveform modulation module comprises a modulation circuit and an inverter, the inverter comprises a plurality of switching devices, and an output terminal of the modulation circuit has a signal connection to the plurality of switching devices in the inverter; and
the modulation circuit is configured to take the target waveform as a modulated wave, and control on/off of the plurality of switching devices in the inverter according to intersection points between the target waveform and a carrier wave, so that the inverter is capable of outputting the pulse sequence, and the width of the pulse in the pulse sequence is matched with the amplitude of the target waveform.

12. The system of claim 11, wherein a power supply of the inverter is adjustable, and a voltage value of the power supply is matched with an excitation voltage value required for inhibiting the tumor cells.

13. The system of claim 11, wherein the signal generation module is further configured to generate a differential waveform with the target waveform while generating the target waveform, and correspondingly, the modulation circuit is further configured to control the on/off of different switching devices in the inverter according to the intersection points between the target waveform and the carrier wave and intersection points between the differential waveform and the carrier wave, so that the inverter is capable of outputting two pulse sequences, a width of the pulse in one pulse sequence is matched with the amplitude of the target waveform, and a width of the pulse in the other pulse sequence is matched with the amplitude of the differential waveform.

14. The system of claim 9, wherein the filter module comprises a selection circuit and at least one filter circuit, each filter circuit is corresponding to one type of waveform, and the selection circuit is configured to input the pulse sequence to a filter circuit matched with the type of the target waveform in response to a received selection signal.

15. The system of claim 9, wherein the filter module further comprises a transformer that is configured to perform voltage boosting on the electric field signal after filtering to generate the target electric field signal.

16. The system of claim 9, wherein the excitation module comprises an electrode selection channel and at least one group of electrodes, the electrode selection channel is connected to each group of electrodes one by one and is configured to control whether to transmit the target electric field signal to each group of electrodes, the at least one group of electrodes is configured to receive the target electric field signal to form a spatial electric field that acts on the tumor cells.

17. The system of claim 16, wherein the at least one group of electrodes is arranged oppositely along different directions to receive the target electric field signal to form a spatial electric field which is capable of penetrating a tumor region.
